(19) United States
(12) Patent Application Publication
(10) Pub. No.: US 2004/0009158 A1
(43) Pub. Date: Jan. 15, 2004

(54) Promotion of neovascularization using bone marrow-derived endothelial-progenitor cells

(75) Inventors: Mark S. Sands, St. Louis, MO (US); Pampee P. Young, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Washington University

(21) Appl. No.: 10/616,821

(22) Filed: Jul. 10, 2003

Related U.S. Application Data
(60) Provisional application No. 60/395,004, filed on Jul. 11, 2002.

(51) Int. Cl. A61K 48/00

(52) U.S. Cl. 424/93.21

(57) ABSTRACT

The present invention relates to a method of treating congenital protein deficiency in a prenatal or postnatal subject. Said method comprises administering to the subject endothelial progenitor cells, which comprise a gene encoding a functional copy of the deficient protein.

### CROSS REFERENCE TO RELATED APPLICATIONS

This application claims priority from U.S. provisional application Serial No. 60/395,004, filed Jul. 11, 2002, which is incorporated herein by reference in its entirety.

This invention was made with Government support under NIH Grant #DK53920 and NIH Grant #HL07088 awarded by the National Institutes of Health. The Government has certain rights in the invention.

### FIELD OF INVENTION

The present invention relates to a method of treating a congenital protein deficiency in a subject in need of such treatment. The method comprises administering to the subject endothelial progenitor cells that comprise a gene encoding a functional form of the deficient protein.

### BACKGROUND OF THE INVENTION

Formation of new blood vessels (neovascularization) is essential not only for normal development but for many diseased states as well. In embryos, blood vessel formation proceeds through two distinct processes, vasculogenesis and angiogenesis (Ferrara N. and Alitalo K., Nature Medicine, Vol.5, No. 12, pp.1359, 1999). Vasculogenesis involves in situ differentiation of endothelial precursor cells (angioblasts) into vascular endothelial cells, which then give rise to primitive vascular networks. In contrast, angiogenesis refers to sprouting of new blood vessels from the preexisting ones, and is characterized by migration and proliferation of fully-differentiated endothelial cells (ECs) resident within parent vessels (Isner J. and Asahara T., The Journal of Clinical Investigation, Vol. 103, No. 9, pp.1231, 1999). Furthermore, the sprouting of new blood vessels is highly dependent on soluble factors such as vascular endothelial growth factor (VEGF), fibroblast growth factor (FGF), and granulocyte/monocyte colony stimulating factor (GM-CSF).

Previously, angiogenesis was considered to be the only mechanism that could give rise to new blood vessels in the adult. Some recent evidence suggests that vasculogenesis can occur in postnatal development; however, such blood vessel formation has only been described in the presence of an ischemic tissue or a blood vessel injury. Asahara et al. have reported that endothelial cell (EC) progenitors, isolated from human peripheral blood incorporate into sites of active angiogenesis in animal models of ischemia (Asahara et al., Science, 275: 964, 1997).

Until recently, therapeutic blood vessel formation has been mainly restricted to regulating the rate of angiogenesis, by either enhancing it or reducing it depending on the disorder. For example, angiogenic growth factors, such as VEGF, administered either as recombinant protein or by gene transfer, have been reported to enhance neovascularization in animal models of ischemia (Isner J. and Asahara T., The Journal of Clinical Investigation, Vol.103, No. 9, pp.1231, 1999). Gene transfer of naked DNA encoding for VEGF has also been reported to enhance angiogenesis when extended to human subjects for treatment of critical limb ischemia (Isner J. and Asahara T., The Journal of Clinical Investigation, Vol. 103, No. 9, pp.1231, 1999). On the other hand, inhibition of tumor growth and metastasis requires abolishing or at least reducing angiogenesis of tumor blood vessels. It has been reported that a treatment with antibodies against VEGF resulted in time-dependent reductions in vascular permeability and diameter of tumor blood vessels, eventually leading to their regression (Ferrara N. and Alitalo K., Nature Medicine, Vol.5, No.12, pp.1359,1999).

Overall, angiogenesis has found many applications in treatments of various disorders. The same can not be said for vasculogenesis, which has been minimally utilized for therapeutic purposes. A potential for therapeutic neovasculogenesis is exemplified in U.S. Pat. No. 5,980,887. It describes methods of using EPCs to treat injured blood vessels and conditions such as pulmonary or limb ischemia, based on the ability of endothelial cell progenitors to migrate to injured or ischemic tissues and differentiate into endothelial cells. However, the methods described therein are not of general applicability due to the fact that endothelial progenitors were only found to incorporate into ischemic sites.

Congenital protein deficiencies result from deficiencies of proteins that are important for normal functioning of the body. The congenital deficiency generally results from the absence, misfolding or any other defect in the protein that renders it incapable of performing its function. For example, one group of such congenital disorders includes blood protein disorders, which are generally characterized by a single gene/protein deficiency. Examples of blood protein disorders include hemophilia A, hemophilia B, von Willebrand disease, α₁-antitrypsin deficiency, antithrombin III deficiency, and other disorders resulting from deficiencies of coagulation proteins. Another group of congenital protein deficiencies includes lysosomal storage diseases, as exemplified by Gaucher's disease, mucopolysaccharidosis type VII (MPS VII), Fabry disease, MPS I, Niemann-Pick disease, Farber disease, and Pompe disease.

Blood protein disorders are characterized by a single gene defect, thereby giving rise to a deficiency in the protein encoded by said defective gene. Hemophilia, a representative blood protein disease, is an X-linked bleeding disorder resulting from a deficiency in blood coagulation factor VIII (hemophilia A) or factor IX (hemophilia B), normally expressed by endothelial cells and hepatocytes, respectively (Kadhom et al., Thrombosis and Haemostasis, Vol. 59, pp.289-294, 1988). The disease is characterized by frequent hemorrhaging episodes, mostly into joints and soft tissues but can also occur in spaces such as intracranial space or retroperitoneal space where it can be rapidly fatal (High, K A, Circ Res, Vol. 88(2)137-144, 2001). Hemophilia is classified as mild, moderate, or severe depending on the circulating levels of the clotting factor; severe disease is defined as <1% of normal levels, moderate as 1-5%, and mild as >5% (High, K A, Circ Res, Vol. 88(2)137-144, 2001). Current treatments for hemophilia consist of intravenous infusions of either plasma-derived or recombinant clotting factors (High, K A, Circ Res, Vol. 88(2)137-144, 2001). Unfortunately, there are many problems associated with these treatments, inclusive of high costs and a need for constant and difficult intravenous (IV) infusions. Furthermore, the IV treatment is generally provided after evidence of hemorrhage, rather then before such an episode occurs. Most importantly, certain plasma preparations (e.g. cryoprecipitate) cannot be virus-inactivated (Manucci P M, Blood, Vol. 97, No. 7, pp. 1915, 2001), leading to transmission of many blood-borne diseases. At present, the leading causes of mortality among patients afflicted with hemophilia are HIV-related disease and end-stage liver disease, which is often times caused by hepatitis viruses.

Von Willebrand disease (vWD) is a common autosomally-inherited disease caused by the deficiency or dysfunction of von Willebrand factor (vWF). Two major functions of vWF are to stabilize factor VIII in plasma and mediate adhesion of platelets at sites of vascular injury (Manucci P M, Blood, Vol. 97, No. 7, pp.1915, 2001). Von Willebrand factor is found in endothelial cells and megakaryocytes as a large, multimeric glycoprotein, and it is constitutively secreted from endothelial cells, which also store a fraction of it in cytoplasmic granules known as Weibel-Palade bodies. Von Willebrand disease displays multiple clinical phenotypes depending on whether the defect lies in the synthesis or storage of vWF. For the patients afflicted with this disease, two main options of treatment comprise desmopressin and transfusions with plasma factors. Desmopressin is a synthetic analogue of vasopressin and it induces vWF secretion into the plasma (Manucci P M, Blood, Vol. 97, No. 7, pp.1915, 2001). Despite the fact that desmopressin is a relatively inexpensive drug, it does not constitute a satisfactory treatment. The drawbacks include the need to administer the drug frequently, ineffectiveness of the drug in patients with certain forms of vWD, and development of resistance to the drug in a significant number of patients. The transfusion therapy carries with it the same complications that are observed in patients with hemophilia.

The lysosomal storage diseases are inherited disorders frequently caused by a deficiency in a single lysosomal enzyme expressed in many different cell types. In contrast to blood protein disorders that result from defective proteins which are secreted into extracellular environment by multiple cell types, lysosomal storage diseases have defects in particular lysosomal enzymes, which are located in intracellular vesicles known as lysosomes. The importance of lysosomal enzymes can be appreciated in light of the fact that they play a role in hydrolysis/digestion of metabolic products that have been targeted to lysosome for degradation. The phenotypes of lysosomal diseases can range from mild, with almost no evidence of lysosomal storage, to multi-system involvement and early mortality (Daly T M and Sands M S, Exp. Opin. Invest. Drugs, 7(10)1-10, 1998). For most of these disorders, the genetic defects are known and the defective genes have been cloned; however, this knowledge has not led to discovery of successful therapies. Currently, the treatments include enzyme replacement or bone marrow transplantation. A partial success has been achieved by intravenously administering purified glucocerebrosidase to patients with Gaucher's disease. However, the studies in animal models of lysosomal storage disease have shown that the effects of enzyme replacement therapy are short-lived, thus requiring frequent enzyme administration, and have limited applicability in cases with CNS involvement since the injected enzyme is not able to cross the blood-brain barrier.

Fabry disease, an X-linked lysosomal storage disease, results from the deficient activity of the enzyme α-galactosidase A, whose lack in turn results in progressive accumulation of globotiaosylceramide and related glycosphingolipids, particularly in the walls of blood vessels. Premature death is associated with vascular conditions of the heart, kidneys, and brain. Mucopolysaccharidoses are a group of lysosomal storage diseases caused by the deficiency of an enzyme required for the normal degradation of glycosaminoglycans. Patients with mucopolysaccharidosis generally exhibit widespread disease, with skeletal and central nervous system (CNS) involvement and spleen and liver enlargement. Niemann-Pick disease encompasses a group of diseases, which are caused by the deficiency of a specific enzyme activity, acid sphingomyelinase (ASM). The three commonly recognized forms of the disease are Types A, B, and C. The disease exhibits multi-organ involvement, resulting in premature death in many cases.

The above-mentioned diseases are outlined briefly herein as some of the examples of congenital protein deficiencies. However, it should be noted that other disorders belonging to this group still present major challenges in terms of therapy. Hence, a need exists to develop novel methods of treatment that would either cure or ameliorate the detrimental effects of congenital protein disorders described herein.

### SUMMARY OF THE INVENTION

Accordingly, among the various aspects of the present invention is the provision of a method for treating a congenital protein deficiency in a prenatal or postnatal subject. The method comprises administering to a subject in need of treatment endothelial progenitor cells that comprise a gene encoding a functional form of the protein responsible for said congenital deficiency. The congenital deficiencies may be do to a partial or complete absence of protein, or at least one mutation in the gene encoding said protein that reduces its functionality.

Other objects and features will be in part apparent and in part pointed out hereinafter.

### BRIEF DESCRIPTION OF THE FIGURES

FIG. 1 depicts low level hematopoietic engraftment following IV administration of bone marrow cells without prior cytoablation. Bone marrow from a representative mouse who received 5×10⁶ cells/g of syngeneic bone marrow is shown at birth (a) or at 4 weeks (b) in the absence of any preconditioning radiation (see Example 1 for details). The cells were incubated with the fluorogenic GUSB substrate C12FdGIcU followed by labeling with anti-mouse CD45PE-conjugated antibody. (c) shows a newborn mouse given equivalent number of GUSB-positive bone marrow cells after receiving 200 rads of radiation. Quadrants were set to exclude greater than 99.9% of events in similarly stained nontransplanted GUSB-deficient mice (d). The number in the bottom left of the upper right quadrant denotes the average engraftment of the animals analyzed. Three animals were analyzed for each nonablative transplantation condition.

FIG. 2 shows that donor-derived cells colocalize with endothelial cell markers in association with vascular structures. Fluorescent immunostaining with anti-GUSB and anti-vWF antibodies in a GUSB-deficient recipient mouse heart (a-c, g-i) and liver (d-f, j-l) obtained 2 weeks (a-f) or 8 weeks (g-l) after administration of syngeneic GUSB-positive bone marrow cells at birth. Indirect fluorescent immunostaining for vWF and GUSB shows that colocalization is evident after 2 weeks and persists two months after administration. Ten micrometer-thick cryosections were labeled with both anti-GUSB and anti-vWF antibodies and detected with fluorescently-tagged secondary antibodies (X40). Immunohistochemistry using anti-vWF and histochemistry for GUSB (red) were performed to demonstrate colocalization by a second method (m-o). Heart tissue obtained from newborn GUSB-deficient mice sacrificed 2 weeks after bone marrow administration was incubated with biotin-conjugated anti-rabbit secondary antibody alone (m) or after incubation with a rabbit polyclonal anti-vWF primary antibody (n) followed by visualization with streptavidin-linked alkaline phosphatase kit (Vector Labs). The sections were then treated with low pH buffer and incubated with GUSB substrate for histochemical analysis of GUSB. A similar dual-stained procedure was performed with BS-1 lectin and GUSB histochemical staining (o). Thick arrows denote GUSB-positive cells that colocalize with vWF- or BS-1 lectin-positive cells. Thin arrows denotes GUSB-positive cells that do not colocalize with vWF expressing cell (X40). See Example 1 for a detailed description of procedures.

FIG. 3 shows that GUSB specific activities (nanomoles of substrate cleaved per hour per milligram of protein) are increased in several tissues when GUSB-positive bone marrow cells are coadministered with VEGF intravenously to newborn GUSB recipients as described in Example 2. The increase is evident both 2 weeks (a) and after 8 weeks (b) after administration. An asterisk indicates a significant (P<0.05) increase in specific activity over recipients not receiving VEGF.

FIG. 4 shows that VEGF increases both newborn recipient vascularity and donor EPC engraftment. Morphometric analysis as described in Example 2(a) of both GUSB-deficient and wildtype newborn mouse recipient heart and liver was performed in the absence (No VEGF) or presence of 2.5 ng/newborn mouse of VEGF (VEGF) by fluorescent immunostaining with anti vWF. (b) anti-vWF antibody used in immunofluorescent analysis to detect endothelial cells on cryosections obtained 2 weeks after administration from heart show increased staining in the presence of VEGF. (c) shows the numbers of vWF-positive cells that colocalized with GUSB-positive cells in the absence or presence of VEGF. An asterisk (*) indicates a statistically significant change over recipients not receiving VEGF at P<0.05.

FIG. 5 depicts VEGF-increased donor EPC engraftment in liver in the sinus area. Indirect fluorescent immunostaining of endothelial cells by anti-vWF and for donor cells by anti-GUSB as described in Example 2 shows colocalization in liver of recipients. Cryosections were obtained from liver of mice at 8 weeks post administration of 5×10⁶ bone marrow at birth. Regions of colocalization are denoted with arrows (x40).

### DETAILED DESCRIPTION OF THE INVENTION

In accordance with the present invention, Applicants have discovered that donor bone marrow-derived EPCs incorporate into the blood vessels of the recipient's normal, nonischemic tissues during the postnatal period. Prior to this finding, EPCs were thought to incorporate only into vascular tissues during early embryonic stage of development, or into vascular tissues of adult animals that are ischemic or have suffered an injury during postnatal development. Thus, as a result of the applicants' discovery, EPCs may be used in cellular and gene therapy of multiple congenital protein deficiencies, including blood protein disorders and lysosomal storage diseases.

Although the deficient enzymes/proteins responsible for congenital protein disorders described herein may be manufactured in cell types other than endothelial cells, the amelioration of a disease can be achieved, at least in some instances, by providing even a small, but continual supply of the deficient protein into the blood stream. It is generally known that for many of the protein deficiencies, only a small amount of the functional copy of a deficient protein is needed to ameliorate or even cure the disease. For example, in hemophilia A, a small amount of the Factor VIII provides protection from any lethal hemorrhage (Walsh, C E, Current Opinion in Pediatrics, Vol.14, pp. 6-12, 2002). A further example is severe combined immunodeficiency (SCID) resulting from the deficiency of adenosine deaminase (ADA) enzyme, wherein the patients that exhibit incomplete deficiency of ADA and express a small fraction of the enzyme are symptom-free (Zanjani E D and Anderson W F, Science, Vol. 285, pp.2084-2088, Sep. 24, 1999). In addition, it is known that even the deficiencies of intracellular proteins can be treated by intravenous administration of functional forms of the deficient proteins. For instance, a recombinantly-produced glucocerebrosidase is administered intravenously to patients with type I Gaucher disease, resulting in improvement of disease-associated symptoms. In accordance with disease improvement, histological examination of patients receiving such therapy has shown that lysosomes decrease in size following administration of glucocerebrosidase, indicating that the recombinant enzyme is able to reach lysosomes and function properly (Desnick et al., Contributions to Nephrology, Vol.136, pp. 174-192, 2001).

Accordingly, one aspect of the present invention is the provision of a method for treating congenital protein deficiencies. In contrast to the current therapies that are life-long, expensive, and not always satisfactory, the method of treatment described herein offers to restore a normal function of a deficient protein responsible for the congenital deficiency by utilizing endothelial progenitor cells. The therapy is based on the fact that EPCs, upon engrafting into blood vessels, can secrete a functional copy of the defective protein directly and continually into the vascular system, thereby allowing the biologically active form of the protein to circulate throughout the body and function at appropriate locations. Even though a deficient protein may be expressed in cells other than endothelial cells in the absence of the congenital deficiency, expression and secretion of the functional form of the deficient protein by endothelial cells alone can provide a sufficient amount of the functional protein to either cure, ameliorate, or inhibit said congenital deficiency.

The endothelial progenitor cells used in the methods described herein can either be autologous cells, modified ex vivo to contain a functional form of the gene encoding for a deficient protein; or they can be heterologous cells, isolated from a subject with a functional form of the deficient protein.

Autologous EPCs can be obtained from a patient's peripheral blood, bone marrow or umbilical cord blood. EPCs can be isolated using antibodies that specifically recognize EPC antigens on immature human hematopoietic progenitor cells (HSCs). For example, antibodies against CD34 and Flk-1 can be used to isolate EPCs from a population of HSCs. Methods for obtaining HSCs are disclosed in, e.g., U.S. Pat. No. 5,199,942. Furthermore, EPCs can also be isolated directly from the peripheral blood or umbilical cord blood, and expanded ex vivo. See, for example, Kalka et al., PNAS, Vol. 97(7), pp.3422-3427, Mar. 28, 2000, and Boyer et al., J Vasc Surg, 31(1 Pt 1), pp.181-189, January 2000.

Isolation of heterologous EPCs can be performed in the same manner described for autologous cells, with a distinction that the heterologous cells are isolated from a healthy donor rather than from a patient. The healthy donor herein refers to a subject who expresses a functional form of the protein that is deficient in the subject to be treated, and who has no diseases that could be transmitted by administration of his/her cells to the afflicted or predisposed patient, such as, for instance, Hepatitis B. In one embodiment, it is desirable that the donor is MHC-matched to the patient who is being treated with the donor's EPCs. MHC-matching can be performed using a mixed lymphocyte reaction (MLR) or a cytotoxic T-cell assay (CTL assay).

Following isolation, EPCs, either autologous or heterologous, are modified in culture to express the functional version of a gene coding for a defective protein. It should be noted, however, that heterologous EPCs do not need to be modified if the protein that is deficient in a patient to be treated is expressed and secreted by endothelial cells of the healthy subject, whose EPCs are used as a source of heterologous cells. For example, treating a patient with Hemophilia A with heterologous cells does not require genetically-modifying said heterologous cells since they express and secrete Factor VIII, the protein that is deficient in patients with Hemophilia A. However, even in such cases, where heterologous endothelial cells secrete a protein that is deficient in a patient with the congenital protein deficiency, it may be desirable to transfect heterologous EPCs with a gene encoding a functional form of the patient's deficient protein in order to increase the expression and secretion of said protein upon EPC engrafment.

The genetic material, such as a gene encoding a functional form of the deficient protein can be introduced into endothelial progenitor cells by any method that will ultimately result in the expression of said genetic material by the ECs. The methods include vectors, liposomes, electroporation, microinjection, coprecipitation with calcium phosphate, naked DNA, adjuvant-assisted DNA, catheters, gene gun, and any other methods available in the art. One skilled in the art can readily apply the methods of recombinant DNA technology to introduce foreign genetic material into the EPCs. The procedures for manipulating nucleic acid sequences disclosed herein are known to those skilled in the art. See generally Fredrick M. Ausubel et al. (1995), “Short Protocols in Molecular Biology,” John Wiley and Sons, and Joseph Sambrook et al. (1989), “Molecular Cloning, A Laboratory Manual,” second ed., Cold Spring Harbor Laboratory Press, which are both incorporated by reference. Additionally, genetic manipulation of cells by introduction of exogenous DNA or RNA for the purpose of expressing a polypeptide or a protein in vivo for the treatment of disease or deficiency in humans or animals has generally been disclosed in U.S. Pat. No. 5,399,346.

If the sequence of the deficient protein or the corresponding gene is unknown, the gene encoding a functional copy of the deficient protein is isolated from the cells of the donor's tissue, blood, or other body fluids. The validity of the isolated gene can be checked by inserting it into a vector containing appropriate regulatory sequences for expression of a gene, and then screening it by conventional methods such as enzymatic digestion, Northern blotting, or Western blotting if the expression product is known. Alternatively, if the DNA sequence of the desired gene is known or the amino acid sequence of the functional protein is known, the gene can be generated by synthetic methods such as by using a DNA synthesizer (Applied Biosystems).

Once isolated, a functional structural gene, such as a DNA sequence coding for a Factor VIII protein, can be modified for expression in vivo by linkage to suitable control regions, such as promoters. Other genes include, but are not limited to, Factor IX, von Willebrand factor, glucocerebrosidase, a-galactosidase A, and acid sphingomyelinase. In the present invention, a promoter is selected that is functional in endothelial cells in order for the gene to be expressed in ECs. For example, a desired gene can be placed under the control of a vascular endothelium-specific promoter, VEGF receptor 2 (VEGFR-2). For description of VEGFR-b 2l , see, for example, Morishita et al., Journal of Biological Chemistry, Vol. 270, no. 46, pp.27948-27953, Nov. 17, 1995). Additional promoters, including but not limited to tie-1 and tie-2, may also be used (see, e.g., Marchetti et al., J Cell Sci, 115(Pt 10):2075-2085,May 15 2002 and Schlaeger et al., Proc Natl. Acad. Sci. USA, Vol. 94, pp. 3058-3063, April 1997, respectively). The linkage of a gene to a promoter can be performed by established protocols for use of expression control sequences including, for example, promoters, operators, and regulators. These protocols are known and routinely used in the art. See, e.g., Current Protocols in Molecular Biology (Ausubel, F. M. et al., eds.), Wiley Interscience, New York. The ability of a promoter and/or other regulatory sequences to direct expression of a gene can be checked by determining the expression of a protein encoded by said gene in appropriate cells. For purposes of the present invention, the appropriate cells can be either endothelial cell lines or primary endothelial cells. The protein expression can be determined, e.g., by sequencing, Northern and Western blotting, or polymerase chain reaction (PCR). Once a gene that is functionally linked to appropriate expression sequences has been obtained, it can be transfected into EPCs using any of the previously-mentioned methods.

In one embodiment, viral vectors are used to transfect EPCs, wherein the viral vectors comprise adenoviral vectors, adeno-associated virus vectors, retroviral vectors, and lentiviral vectors. An adenoviral vector may include essentially the complete adenoviral genome (Shenk, et al., Curr. Top. Microbiol. Immunol., 111:1-39,1984). Alternatively, the adenoviral vector may be a modified adenoviral vector in which at least a portion of the adenoviral genome has been deleted. Adenoviral vectors may be produced according to He, et al. (PNAS 95:2590-2514, 1998; Chartier, et al., J. Virol. 70:48054810,1996 and Hitt, et al., Methods in Molecular Genetics, 7:13-30, 1995). Methods of transferring genes into cells using adenoviral vectors has been described in PCT/US95/15947. Furthermore, a number of adenoviral vectors have been developed for the transduction of genes into cells (Berkner, et al., BioTechniques 6:616-629,1988). Importantly, constitutive high level expression of the transduced gene products has been achieved in adeno vectors.

Retroviral vector production and use are also known per se. See for example, U.S. Pat. No. 5,910,434, Veres, et al., J. Virol., Vol. 72:1894-1901, 1998; Agarwal, et al., J. Virol., Vol. 72:3720-3728, 1998; Forestell, et al., Gene Therapy, 4:600-610, 1997; Plavec, et al., Gene Therapy, 4:128-139, 1997; Forestell, et al., Gene Therapy, 2:723-730,1995; and Rigg, et al., J. Virol., 218:290-295, 1996. The genome of recombinant retroviral vector is comprised of long terminal repeat (LTR) sequences at both ends which serve as a viral promoter/enhancer and a transcription initiation site and a Psi site which serves as a virion packaging signal and a selectable marker gene. An example of such vector is pZIP NeoSV (Cepko, et al., Cell 53:103-1062, 1984). A gene encoding a functional form of the protein responsible for a congenital protein deficiency can be cloned into a suitable cloning site in the retroviral genome. Retrovirus vectors, suited ideally for ex vivo gene transfer, can mediate long term (months to years) transgene expression due to viral integration into the host genome. A number of retroviral vectors is disclosed in, e.g., U.S. Pat. Nos. 5,672,510, 5,707,865, and 5,817,491.

Adeno-associated virus vectors are disclosed, for example, in U.S. Pat. Nos. 5,139,941, 5,436,146, and 5,622,856. These virus vectors have the advantage of efficient transduction in vivo as well as the potential for long term transgene expression (months) due to viral integration into the host genome.

Lentiviral vectors which can also be used in gene therapy are disclosed, e.g., in U.S. Pat. Nos. 5,665,577, 5,994,136, and 6,013,516. In addition to viral vectors, synthetic vectors may be used, which are disclosed, for example, in U.S. Pat. Nos. 4,394,448, and 5,676,954.

In general, preparation of vectors of can be accomplished by procedures disclosed herein and by recognized recombinant DNA techniques, e.g. preparation of plasmid DNA, cleavage of DNA with restriction enzymes, ligation of DNA, transformation or transfection of a host, culturing of the host, and isolation and purification of the desired vector construct. Such procedures are generally known and disclosed e.g. in Sambrook et al., Molecular Cloning (2d ed. 1989); and Ausubel et al., Current Protocols in Molecular Biology, John Wiley & Sons, New York, 1989.

In a further embodiment of the present invention, autologous or heterologous EPCs may be modified by introducing nucleic acids into said EPCs, wherein said nucleic acids encode proteins that enhance survival and/or differentiation of EPCs. For instance, a nucleic acid encoding VEGF can be introduced into EPCs. The introduction of nucleic acids into EPCs can be performed by any available means mentioned above, such as vectors, liposomes, naked DNA, catheters, gene gun, and the like.

A subject to be treated with the method of the present invention can be any mammal, and preferably is a human. Either autologous or heterologous endothelial progenitor cells may be used, however, it is preferable to use autologous cells if they can be obtained in sufficient quantity.

In one embodiment, a patient is treated post-natally, at any stage at which non-pathologic vasculogenesis is still continuously occurring. It is desirable that the patient is treated early during postnatal development due to the fact that at such time EPC incorporation is considerably higher than later in life. Furthermore, it is desirable to treat patients before the onset of irreversible pathology, such as pathology that is seen in the central nervous system. Accordingly, a patient is preferably treated during juvenile stage (12-15 years of age), more preferably during childhood stage (6-10 years), still more preferably during early childhood stage (1-5 years), and even more preferably during infancy (<1 year).

The route of administration may be selected from the group comprising intravenous, intramuscular, intraperitoneal, or any other appropriate route. In a preferred embodiment, the route of administration is intravenous.

Administration of cells has previously been used in mammals including humans for therapeutic purposes, e.g., in bone marrow transplantation, and is well known in the art. Since BM cell administration is homologous to EPC administration, any of the routine methods used for BM transplantation may also be used to deliver EPCs. For example, bone marrow (BM) transplantation in mice with a lysosomal strorage disease, such as Fabry disease, has been described in, e.g., Qin et al., PNAS, Vol.98, no. 6, 3428-3433, 2001. Furthermore, an example of BM cell transplantation in an infant afflicted with Farber disease (ceramidase deficiency) can be found in Yeager et al., Bone Marrow Transplant., 26(3):357-363, 2000.

It is generally known that transplantation of foreign tissue into a recipient may be accompanied by rejection of said transplanted tissue. Accordingly, the use of heterologous cells could have potential detrimental effects for a recipient if there is a strong reaction of the recipient's immune system against the donor's EPCs. This reaction could lead to the destruction of heterologous EPCs or even to the death of the recipient. To avoid any negative effects, immunosuppressive drugs may be used to supplement the transplantation of heterologous cells. Their use prevents, or at least minimizes the adverse actions of the immune system. The immunosuppressive drugs commonly used in transplantation include but are not limited to Cyclosporine A, prednisone, methyl prednisolone, azathioprine, cyclophosphamide, antilymphocyte globulin, and antithymocyte globulin.

Generally, Cyclosporine A (CSA) treatments can provide sufficient immunosuppression to prevent rejection of the donor tissue. CSA treatment protocols to prevent transplant rejection are known in the medical field. Furthermore, the agents thought to induce tolerance to transplanted tissue, such as, e.g., CTLA-4lg can be administered to recipients (Lin et al. (1993), J Exp Med 178:1801-1806). It should be noted, however, that any clinically used immunosuppressive drugs may be used to prevent rejection of heterologous cells. In one preferred embodiment, the immunosuppressive drug comprises Cyclosporin A.

In another embodiment, a subject is treated prenatally, in utero. A family history is generally a strong indication to test as early as possible for the presence of a congenital protein deficiency in a neonate. In utero treatments are desirable since it is believed that mammals are “immunotolerant” at prenatal stage of development, i.e. their immune system will not so readily attack foreign genes. In addition to their potential immunotolerance, treating prenatal fetuses is advantageous since the treatment can begin before the congenital deficiency-related symptoms appear. Furthermore, the success of in utero transplants has been confirmed in recent years by their effective use in treatments of severe combined immunodeficiency syndrome in humans.

In utero treatment using EPCs is further supported by a number of existing clinical protocols that are based on ex vivo retroviral vector transduction of HSCs, which are inserted into a patient following genetic modification. See, for example, Maloch et al., Proc. Natl. Acad. sci. USA, 94, pp.12133, 1997, Liu et al., Hum. Gen. Ther., 8, pp.1715, 1997, Dunbar et al., Blood, 85, pp.3048, 1995, Hanania et al., Proc. Natl. Acad. Sci. USA, 93, pp.15346, 1996, Bardignon et al., Science, 270, pp.470, 1995, Dunbar et al., Hum. Gen. Ther., 7, pp. 231, 1996, and Kohn et al., Nature Med., 1, pp.11017, 1995. The use of EPCs is more advantageous than the use of HSCs in that it does not require cytoablation which is frequently performed prior to administration of genetically-engineered HSCs.

To make in utero treatments practical, a subject needs to be diagnosed during prenatal stages of development. To this effect, advances in prenatal diagnosis and molecular biology now allow the identification of numerous congenital protein deficiencies by evaluation of trophoblastic tissue obtained by chorionic villus sampling (CVS) at 8 to 10 weeks of gestation or by amniocentesis, which can be performed as early as 13 weeks of gestation. Furthermore, high-resolution ultrasound and midgestational interventional techniques have developed to the point that the manipulative techniques necessary to carry out second trimester treatment at low risk to the mother and fetus are now available. Thus, a prenatal subject, upon diagnosis of a congenital protein deficiency, can be treated with EPCs as early as second trimester. EPCs can be administered to the fetus either intravenously or intraperitoneally, via in utero injection. For a more detailed review of in utero treatments, see, e.g., Zanjani E D and Anderson W F, Science, Vol. 285, pp.2084-2088, Sep. 24, 1999.

The number of cells to be administered depends on multiple factors such as seriousness of condition, the age of the patient, presence of additional conditions, etc. In addition, the dosage may vary within the effective range depending upon the dosage form employed and the route of administration utilized. The exact formulation, route of administration and dosage can be chosen by the individual physician in view of the patient's condition. Preferably, the amount of endothelial progenitor cells administered to the patient is between about 10⁶ to about 10¹⁸. More preferably, the number of cells is between about 10⁸ to about 10¹⁵, and even more preferably, the number of cells is between about 10⁹ to about 10¹².

Applicants have further found that incorporation of donor bone marrow-derived EPCs into a newborn neovasculature can be enhanced by use of additional therapies. Thus, in a further embodiment, EPCs can be administered in combination with another therapy. For example, radiation therapy prior to EPC administration can significantly increase the incorporation rate of EPCs into vascular tissues, improving the efficacy of the EPC treatment. Ablation of recipient patient bone marrow cells may not be required, but if it is used, it can be accomplished by standard total body irradiation (Kim, et al., Radiology, 122:523, 1977) or by chemotherapy with a variety of commonly used compounds including, but not limited to Busulfan (Tutschka, et al., Blood, 70:1382-1388, 1987), following the conventional methods.

In another embodiment, the endothelial progenitor cells can be administered in conjunction with endothelial cell mitogens. These include but are not limited to VEGF, acidic and basic fibroblast growth factors (aFGF and bFGF respectively), epidermal growth factor (EGF), transforming growth factor α and β (TGF-α and TGF-β respectively), platelet-derived endothelial growth factor, platelet-derived growth factor, tumor necrosis factor α (TNF-α), hepatocyte growth factor (HGF), insulin like growth factor, erythropoietin, colony stimulating factor (CSF), macrophage-CSF (M-CSF), granulocyte/macrophage CSF (GM-CSF), and nitric oxide synthase, of which VEGF is the preferred mitogen. The administration of mitogens can be performed prior to, simultaneously with, or subsequent to EPC administration. The mitogens can be delivered by different routes of administration, such as intravenous, intramuscular, intraperitoneal, or subcutaneous. Furthermore, a nucleic acid encoding for the mitogen can be used instead of the recombinant protein. The nucleic acid can be administered to the patient by different means including, for example, injection into a desired tissue or gene gun. For the methods of administering endothelial cell mitogens either as recombinant proteins or nucleic acids, see, for example, Isner J. and Asahara T., The Journal of Clinical Investigation, Vol.103, No. 9, pp.1231, 1999.

In an alternative embodiment, the methods described herein that utilize autologous or heterologous endothelial progenitor cells may also be performed with autologous or heterologous bone marrow (BM) cells due to the fact that bone marrow cells comprise EPCs. Isolation and manipulation of BM cells are well known in the art. See, generally Lasky, Larry C., ed., Warkentin, Phyllis, ed., Marrow and Stem Cell Processing for Transplantation, American Association of Blood Banks, Bethesda, Md., 1995, and the references listed above. It should be noted that for treatments that utilize bone marrow cells, it is important to determine the number of EPCs contained in said BM cells. Such fraction determination allows to calculate the number of bone marrow cells that needs to be administered in order to deliver the effective amount of EPCs. One of ordinary skill in the art can easily determine the number of EPCs in bone marrow cells and the consequent amount of BM cells to be administered. Genetic modification and administration of bone marrow cells can be performed in the same manner as described above for EPCs.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

Abbreviations and Definitions

To facilitate understanding of the invention, a number of terms are defined below:

The term “biologically active” as used herein means that the transgenic protein demonstrates similar properties, but not necessarily all of the same properties, and not necessarily to the same degree, as the natural protein.

“Blood protein disorder”, as used herein, includes any disorder resulting from a defect or deficiency in a protein that circulates in the blood stream or can reach target organs via blood stream. Hence, the blood protein disorders include, but are not limited to hemophilia A, hemophilia B, von Willebrand disease, α₁ antitrypsin deficiency, antithrombin III deficiency, and other disorders resulting from deficiencies of coagulation proteins.

“Congenital protein deficiency”, as used herein, refers to a disorder present at birth, which is due to a deficiency in a particular protein responsible for such congenital protein deficiency, wherein the protein is produced and secreted by endothelial cells in the absence of the congenital deficiency. A protein deficiency may result from: 1) a complete deficiency of the protein, 2) an incomplete deficiency of the protein, or 3) one or more mutations in the gene encoding said protein that reduce its functionality. An incomplete protein deficiency reduces the concentration of the protein in the body, thereby reducing its activity. Mutations that can give rise to deficient proteins include but are not limited to nonsense mutations, stop-codon mutations, base pair deletions, out-of-frame shifts, etc. Such mutations can either reduce the amount of the produced protein, or can abolish the function/activity of a protein.

“Effective amount” as used herein indicates an amount of endothelial progenitor cells that is provided with a goal of achieving either the effect that the diseases/disorders described herein are cured or ameliorated, or if the cells have been given prophilactically, the effect that said diseases/disorders are prevented from manifesting themselves.

“EPC” is an abbreviation for endothelial progenitor cell(s).

“Functional form” or “functional copy” as used herein in connection with the term “protein” refers to a form of the protein that functions in substantially the same or the same manner as the wild-type protein. For example, a functional form of Factor VIII refers to a form of the Factor VIII protein that has substantially the same functions and properties as the wild-type Factor VIII.

“Lysosomal storage disease” as used herein includes any disorder resulting from a defect in a lysosomal enzyme that can be corrected by providing the lacking enzyme. Accordingly, the lysosomal storage diseases include, but are not limited to Gaucher's disease, mucopolysaccharidosis type VII (MPS VII), Fabry disease, MPS I, Niemann-Pick disease, Farber disease, and Pompe disease.

“MHC” as used herein refers to the major histocompatibility complex.

“MPS VII” is an abbreviation for mucopolysaccharidosis type VII.

“MPS I” is an abbreviation for mucopolysaccharidosis type I.

“Subject” as used herein for purposes of treatment includes any human or animal subject afflicted with a congenital protein disorder, and preferably is a human subject. For methods of prevention, the subject is any human or animal subject, and preferably is a human subject who is or at risk for or predisposed to a congenital protein deficiency disorder.

“Treating” and “to treat”, as used herein, mean to alleviate symptoms, eliminate the causation of a blood protein disorder or lysosomal storage disease either on a temporary or a permanent basis, slow the appearance of symptoms and/or progression of said disorder or disease, or prevent said disorder or disease (i.e. to treat prophylactically). The term “treatment” includes alleviation, elimination of causation or prevention of a blood protein disorder, lysosomal storage disease, or other congenital protein deficiency, wherein the protein is expressed and secreted by endothelial cells in the absence of the congenital deficiency.

The following examples illustrate the invention, but are not to be taken as limiting the various aspects of the invention so illustrated.

### EXAMPLES

### Example 1

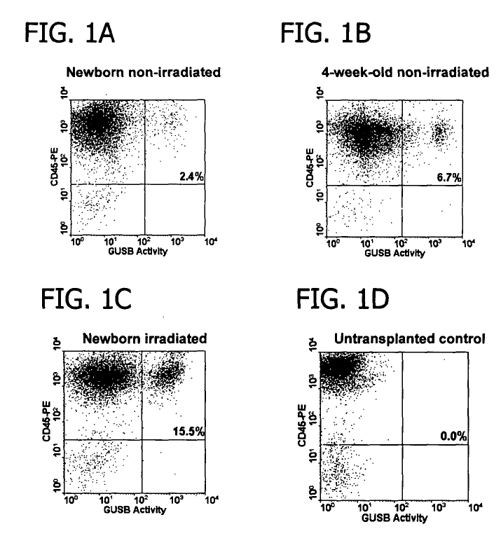

Incorporation of Donor-Derived EPCs into Vascular Structures P Material and Methods: GUSB-deficient homozygous mutant (mps/mps) mice were obtained from a B6.C-H-2^{bm1}/by Birgus^{mps}/+colony maintained by M.S.S. at Washington University (St. Louis, Mo.). Homozygous transgenic donor mice were obtained from a separately bred group of syngeneic animals carrying the human GUSB cDNA as a transgene to increase cellular GUSB expression. Homozygous GUSB-deficient mice were identified at birth by the absence of β-glucuronidase activity using a fluorometric assay on a small sample obtained by toe clipping (FIG. 1). Unfractionated bone marrow (BM) from syngeneic donor mice was obtained by flushing both femurs with PBS supplemented with 0.5% BSA and 1 mM EDTA, according to (15 m). Newborn GUSB-deficient mice were injected intravenously (through the superficial temporal vein) within 3 days of birth with 5*10⁶ nucleated bone marrow cells isolated from syngeneic adult GUSB-positive mice. In parallel, 4-week-old GUSB-deficient mice were also IV injected (through the lateral tail vein) with approximately equal dose per weight (1*10⁸) of unfractionated bone marrow cells isolated from the same donor mice as utilized for the newborn recipients. The cells were injected in a volume of 100 ul per mouse, and administered without any radioablative or chemical preconditioning. 33 newborn and 10 4-week-old GUSB-deficient mice were utilized in this study. At least three animals were studied at each time point.

Histochemistry, immunofluorescence, immnohistochemistry, and morphometry: Tissue sections from organs were harvested after 2, 4, and 8 weeks after BM administration and processed as previously described (Freeman et al., Blood 94, pp. 2142-2150). Histochemical analysis for GUSB activity was performed as described by using naphthol-AS-BI-β-D-glucuronide (ASBI) as the substrate (19m). For immunofluorescence, sections were fixed for 20 minutes at 4° C. in 100% acetone and then incubated with MOM block (5%w/w papain, 2 μM EDTA, pH 7.4, 20 mM L-cysteine) at dilution of 1:20 and PBS blocking buffer (0.01 g/ml BSA, 2 μg powdered milk/ml, 3 μl/ml Triton X-100) and 10% goat serum for 1-2 hours. The slides were then washed with PBS and incubated with rabbit polyclonal antibody against human vWF (Dako, Carpinetria, Calif.) (1:250) and mouse monoclonal antibody against mouse GUSB (gift of W. Sly) (1:100) in PBS-BB/10% goat serum overnight at 4° C. Slides were washed and then incubated with goat anti-mouse and goat anti-rabbit IgG FITC- or Rhodamine-conjugated fluorochromes for 1-2 hours (Calbiochem, San Diego, Calif.). For immunohistochemistry, tissues were fixed as above and processed as described (15, 19m). Anti-vWF or BS-1 lectin were used at 1:100 or 1:50 dilutions, respectively, negative controls in which the tissues were incubated with the labeled secondary antibody alone in the absence of primary antibody were performed in parallel with all experiments. Morphometric quantitation of changes in vascularity in the liver in response to rVEGF was done on seven to fourteen randomly chosen fields (X20) from tissues obtained from at least two experimental animals by fluorescence microscopy using an antibody to vWF as described above. The total number of vWF-positive cells was counted for each image. Analysis of the heart was performed by counting the number of times vWF-positive vessels crossed the lines of a grid superimposed on a digitally-captured image. Quantitation of numbers of GUSB-positive cells that colocalize with endothelial cells expressing vWF was done by counting the numbers of colocalize vessels or cells in 18-30 random section of tissue on at least two different animals. The area of each section was measured and data expressed as numbers of colocalization of GUSB and vWF per square centimeter.

Histopathology: Tissue samples were removed and fixed in 10% neutral-buffered formalin and embedded in paraffin, then sectioned for staining. Immunohistochemistry was performed after deparaffinizing in xylene and washing, followed by antigen retrieval in citrate buffer (pH 6) by microwaving for 8 minutes. Slides were treated with 3% H₂O₂ for 5 minutes, rinsed, blocked with BSA, and then incubated with either anti-vWF (1:100) or with BS-1 lectin (1:160) overnight. Slides were washed and secondary antibody applied and visualized using the LSAB peroxidase kit (Dako, Carpinteria, Calif.). Slides were counterstained with hematoxylin.

Immunolocalization experiments were performed utilizing an antibody that recognizes endothelial cells, anti-von Willebrand factor (vWF) or BS-1 lectin and an antibody identifying donor-derived cells, anti-mouse GUSB.

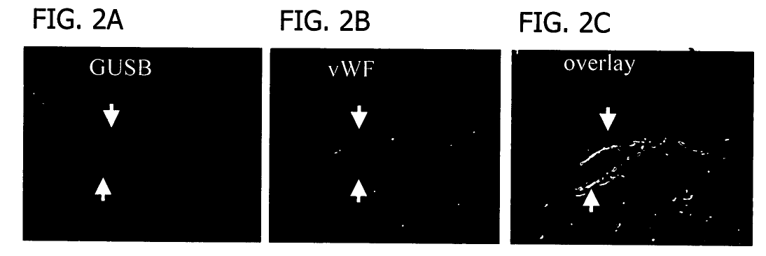

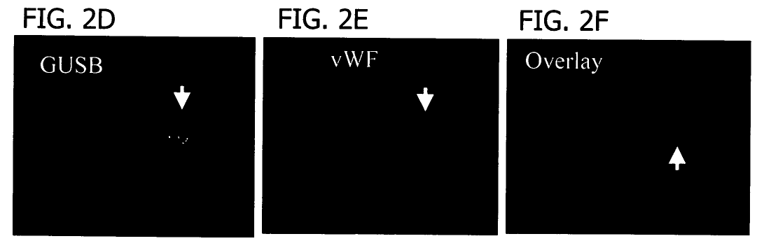

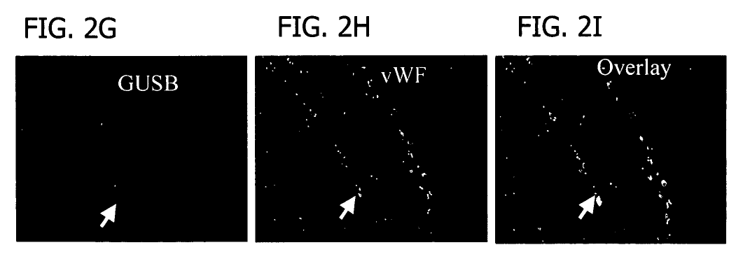

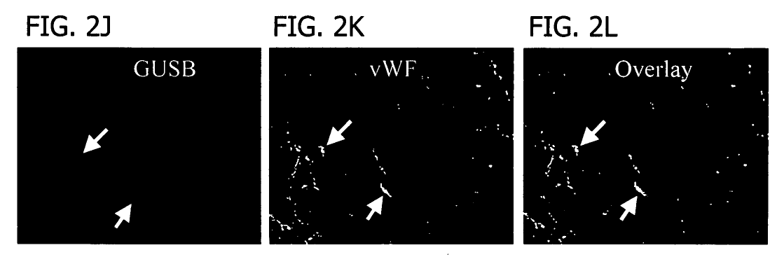

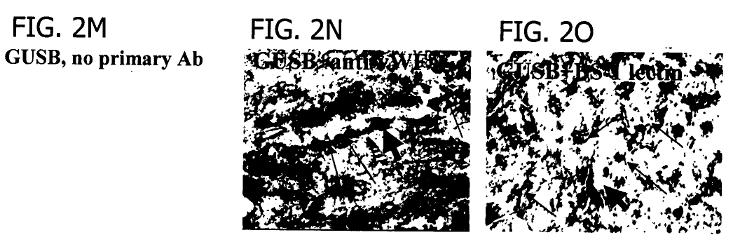

As shown in FIG. 2, fluorescent immunostaining was performed with anti-GUSB and anti-vWF in a GUSB-deficient recipient mouse heart (a-c, g-i) and liver (d-f, j-l) obtained 2 weeks (a-f) or 8 weeks (g-l) after administration of syngeneic GUSB-positive bone marrow cells at birth. Ten μm thick cryosections were stained with both anti-GUSB and anti-vWF antibodies and detected with fluorescently-labeled secondary antibodies. (m-o) show immunohistochemistry using anti-vWF and histochemistry for GUSB (red), both performed to demonstrate colocalization by a second method. Heart tissue obtained from newborn GUSB-deficient mice sacrificed 2 weeks later was incubated with biotin-conjugated anti-rabbit secondary antibody alone (m) or after incubation with a rabbit polyclonal anti-vWF primary antibody (n) followed by visualization with streptavidin-linked alkaline phosphatase kit. The sections were then treated with low pH buffer and incubated with GUSB substrate for histochemical analysis of GUSB. (o) was incubated with biotin-linked BS1 lectin and visualized in a similar manner.

Results: The role of bone marrow-derived EPCs in new blood vessel formation in vivo was monitored by using GUSB-deficient mouse as a recipient and a syngeneic GUSB-positive mouse as a donor of bone marrow cells. GUSB and vWF colocalize in association with vascular structures of the heart and liver of newborn recipients analyzed two weeks after administration (FIG. 2). The numbers of donor-derived cells that colocalize with vWF declined dramatically after 8 weeks (FIG. 2). In general, indirect fluorescent immunostaining for vWF and GUSB shows that colocalization is evident after 2 weeks and persists two months after administration, albeit significantly diminished. Furthermore, areas of colocalization were primarily small capillaries. Analysis of the 4-week-old murine recipients' hearts, livers, and spleens failed to detect any colocalization at all time points analyzed (data not shown). To confirm the finding with another method, immunohistochemistry was performed on frozen sections obtained from heart using either an anti-vWF antibody or BS-1 lectin. The same sections were subsequently treated in an acidic buffer and exposed to the GUSB substrate, naphtol-AS-BI-β-D-glucuronide (ASBI). The cells expressing the enzyme, i.e. donor-derived cells turned red after in situ cleavage of the ASBI substrate (FIG. 2). Control experiments showed that the combination of these techniques lowered the sensitivity of detecting GUSB-positive cells (data not shown). Nevertheless, there were numerous GUSB-positive cells in several sections obtained from newborn heart isolated 2 weeks post administration of bone marrow cells that colocalize with either BS-1 or vWF-positive cells in association with a vascular structure. Due to the technical difficulties, lung and kidney were not assessed for engraftment.

### Example 2

Coadministration of VEGF to Newborns Increases Vascularity and Donor-Derived EPC Engraftment

Materials, methods and all staining protocols are the same as in Example 1 unless otherwise indicated.

GUSB-deficient newborn mice received 1.25 ng or 0.4 ng of rhVEGF164 (R&D Systems) either with or without bone marrow cells at day 1 of birth and a repeat administration of an equivalent dose of rhVEGF164 alone IV on day 3 after birth. VEGF-injected wildtype and saline-injected GUSB-deficient mice were used as controls.

Syngeneic bone marrow cells were administered as described in example 1 either with a low dose (0.4 ng/newborn mouse) or high dose (1.25 ng/newborn mouse) of recombinant VEGF to newborn GUSB-deficient mice on day 1 of birth. The animals were readministered the same dose of VEGF on day 3. Control GUSB-deficient mice were injected with bone marrow cells alone and received a second injection of equal volume of saline on day 3 as the VEGF-treated mice. In parallel, wildtype newborn mice were also injected with either saline, and low or high dose of VEGF on day 1 and 3 after birth. No acute toxicity was observed in recipients administered bone marrow cells, saline, or low dose of VEGF alone or in combination with BM cells. Both GUSB-deficient and wildtype newborns receiving high dose of VEGF required longer time to resuscitate and exhibited mildly prolonged bleeding at the injection site. Systemic administration of VEGF has been known to cause hypotension in other animal models. Importantly, there were no angiomas observed either grossly or on histologic sections in control or VEGF-treated animals. The study was carried out to 8 weeks after coadministration of BM cells and VEGF, and during that time no obvious abnormalities were evident in the recipients.

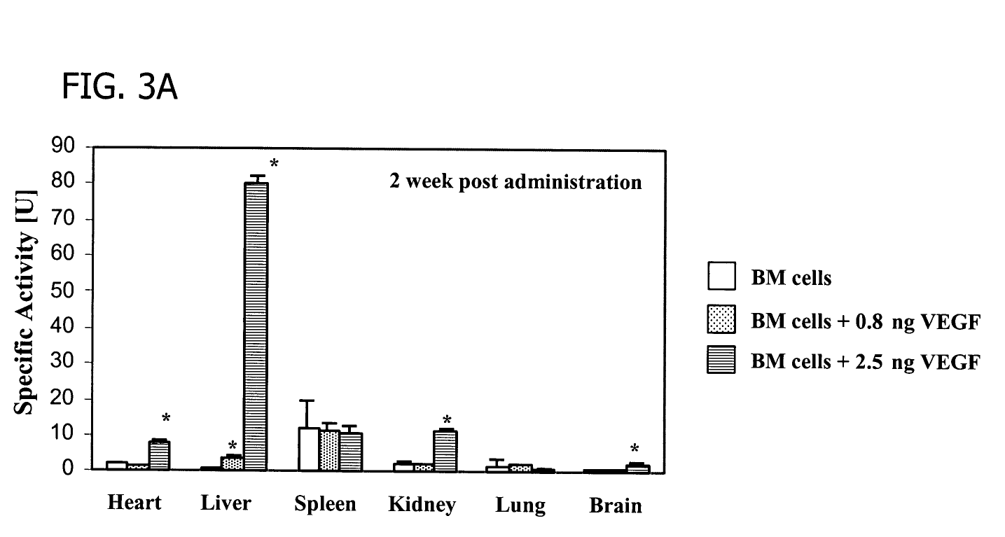

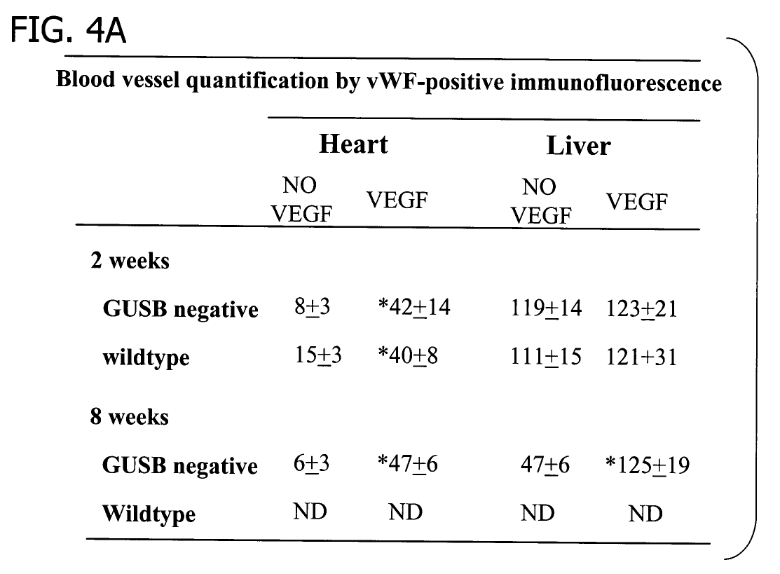

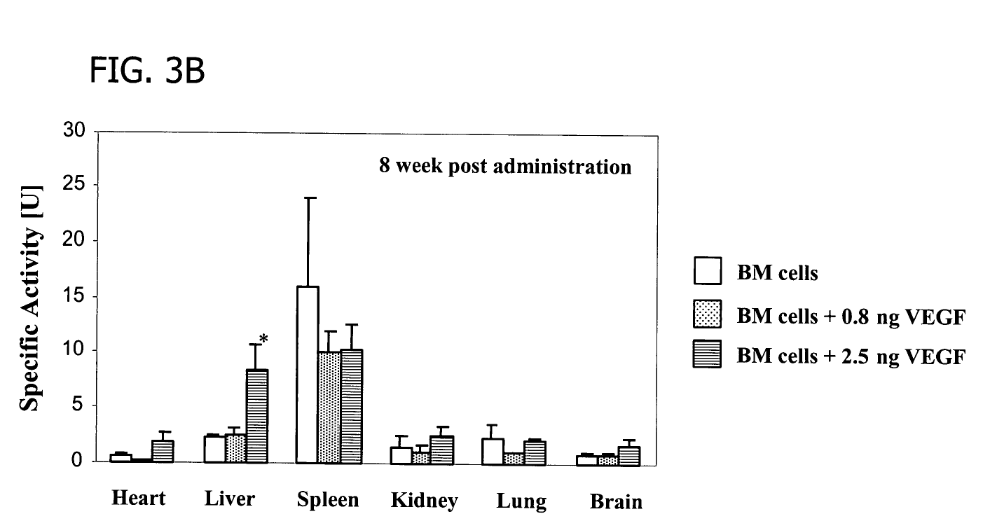

Results: The levels of donor-derived, GUSB-positive cells in various organs were measured by a quantitative biochemical assays for GUSB activity. Enzyme activity was increased in several tissues, including the heart and liver, when bone marrow cells were administered with VEGF (FIG. 3). Furthermore, the increased levels of donor cells in VEGF-treated recipients persisted even after 2 months (FIG. 3b). Although the bone marrow was not analyzed by quantitative biochemistry, quantitative analysis of the spleen suggested no increased donor hematopoietic engraftment in the presence of VEGF when compared to recipients of bone marrow cells alone (FIG. 3).

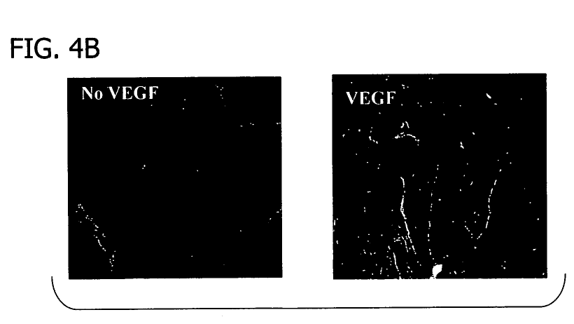

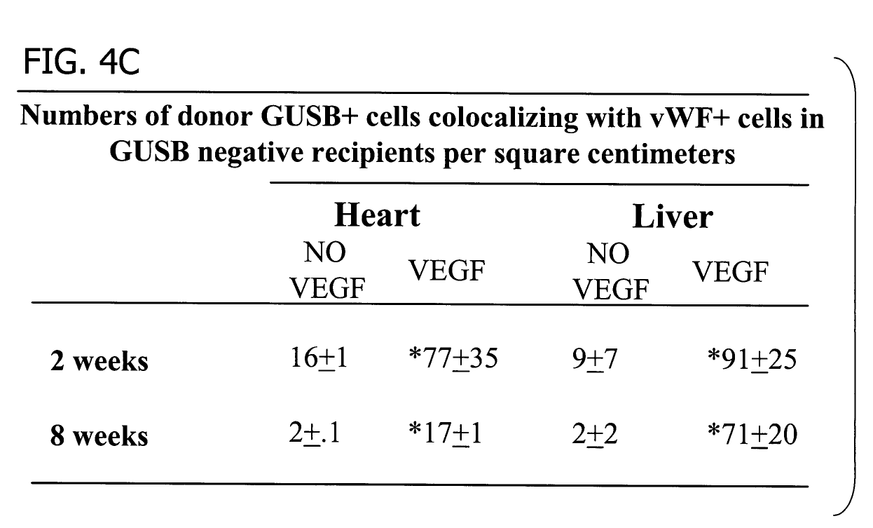

The effect of recombinant VEGF on vascularity was assessed by morphometric measurements of tissues analyzed by immunofluorescence using an anti-vWF antibody. Because the levels of EPC incorporation in the absence of VEGF were greatest in the heart and liver, morphometric analysis was focused on these two organs. Vascular density in the heart was increased significantly by 2 weeks after VEGF administration in both wildtype and GUSB-deficient recipients (FIG. 4a). This increase persisted at 8 weeks post administration. Analysis of the liver showed no difference in the numbers of sinus lining cells between VEGF-treated and untreated animals at 2 weeks in either GUSB-deficient or wildtype animals. In contrast, 8 weeks after administration, VEGF-treated liver had greater numbers of vWF-positive sinus lining cells as compared to untreated animals (FIG. 4b).

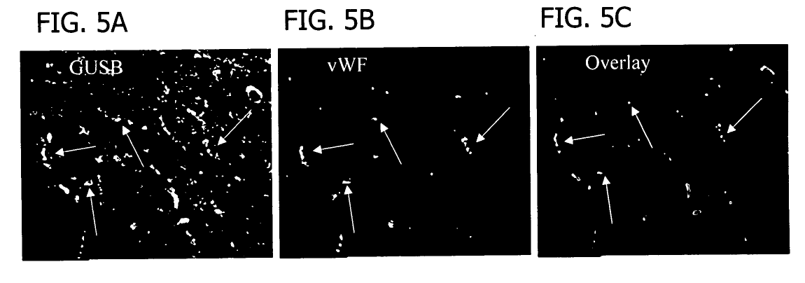

To investigate if VEGF had an effect on the numbers of donor-derived endothelial cells engrafted in newborn recipients, immunofluorescent colocalization studies using anti-vWF and anti-GUSB antibodies were performed on VEGF-treated animals and newborns that only received BM cells. An increased number of donor-derived cells was found in association with vessels of various sizes throughout the heart and liver obtained two weeks after administration. The numbers of vessels containing GUSB-positive endothelial cells were counted and expressed in terms of surface area (FIG. 4c). The distribution of donor-derived endothelial cells in the liver after 2 weeks included larger vessels as well as what appeared to be microvessels located near larger veins. Donor-derived endothelial cells persisted after 8 weeks in both heart and liver (FIGS. 4 and 5). Analysis of liver 8 weeks after administration showed that the donor-derived endothelial cells were found in sinus-lining area (FIG. 5).

Statistical Analysis: Student's t tests were performed to compare different data sets. All data (both in Examples 1 and 2) were presented as mean plus or minus SEM, and a P value of <0.05 was interpreted to denote statistical significance.

What is claimed is:

1. A method of treating a congenital protein deficiency in a subject, said method comprising administering to the subject endothelial progenitor cells that comprise a gene encoding a functional form of the protein responsible for said congenital deficiency at a stage of the subject's life at which non-pathologic vasculogenesis occurs.

2. The method of claim 1, wherein the subject is treated pre-natally.

3. The method of claim 1, wherein the subject is treated post-natally.

4. The method of claim 3, wherein the subject being treated post-natally is still continuously exhibiting signs of non-pathological vasculogenesis.

5. The method of claim 1, wherein the subject is a human.

6. The method of claim 1, wherein the congenital protein deficiency is due to a complete protein deficiency.

7. The method of claim 1, wherein the congenital protein deficiency is due to an incomplete protein deficiency.

8. The method of claim 1, wherein the congenital protein deficiency is due to at least one mutation in a gene encoding the protein, wherein the mutation results in reduced activity of the protein.

9. The method of claim 1, wherein the congenital protein deficiency comprises a blood protein disorder or lysosomal storage disease.

10. The method of claim 9, wherein the blood protein disorder comprises hemophilia A, hemophilia B, von Willebrand disease, α₁-antitrypsin deficiency, or antithrombin III deficiency.

11. The method of claim 10, wherein the blood protein disorder is hemophilia A.

12. The method of claim 10, wherein the blood protein disorder is hemophilia B.

13. The method of claim 10, wherein the blood protein disorder is von Willebrand disease.

14. The method of claim 9, wherein the lysosomal storage disease comprises Gaucher's disease, mucopolysaccharidosis type VII (MPS VII), Fabry disease, mucopolysaccharidosis type I (MPS I), Niemann-Pick disease, Farber disease, or Pompe disease.

15. The method of claim 14, wherein the lysosomal storage disease is Gaucher's disease.

16. The method of claim 14, wherein the lysosomal storage disease is MPS VII.

17. The method of claim 14, wherein the lysosomal storage disease is Fabry disease.

18. The method of claim 14, wherein the lysosomal storage disease is Niemann-Pick disease.

19. The method of claim 1, wherein the endothelial progenitor cells comprise autologous endothelial progenitor cells.

20. The method of claim 19, wherein the autologous endothelial progenitor cells are modified ex vivo prior to administration thereof to the subject in need of treatment, wherein the modification of the cells comprises introducing into said cells a gene encoding a functional form of the protein responsible for said congenital deficiency.

21. The method of claim 1, wherein the endothelial progenitor cells comprise heterologous endothelial progenitor cells.

22. The method of claim 21, wherein the heterologous endothelial progenitor cells are modified ex vivo prior to administration thereof to the subject in need of treatment, wherein the modification of the cells comprises introducing into said cells a gene encoding a functional form of the protein responsible for said congenital deficiency, wherein the deficient protein is not expressed by endothelial cells of other subjects not suffering from the congenital protein deficiency.

23. The method of claim 21, further comprising administering to the subject an immunosuppressive drug.

24. The method of claim 23, wherein the immunosuppressive drug is selected from the group consisting of: Cyclosporine A, prednisone, methyl prednisolone, azathioprine, cyclophosphamide, antilymphocyte globulin, and antithymocyte globulin.

25. The method of claim 24, wherein the immunosuppressive drug is Cyclosporine A.

26. The method of claim 1, further comprising administering to said subject a therapeutically-effective dose of radiation prior to the administration of endothelial progenitor cells.

27. The method of claim 1, further comprising administering to said subject an endothelial cell mitogen.

28. The method of claim 27, wherein the endothelial cell mitogen is selected from a group consisting of: vascular endothelial growth factor (VEGF), acidic and basic fibroblast growth factors (aFGF and bFGF respectively), epidermal growth factor (EGF), transforming growth factor α and β (TGF-α and TGF-β 5 respectively), platelet-derived endothelial growth factor, platelet-derived growth factor, tumor necrosis factor α (TNF-α), hepatocyte growth factor (HGF), insulin like growth factor, erythropoietin, colony stimulating factor (CSF), macrophage-CSF (M-CSF), granulocyte/macrophage CSF (GM-CSF), and nitric oxide synthase.

29. The method of claim 28, wherein the endothelial cell mitogen is VEGF.
